(19) United States
(12) Patent Application Publication
(10) Pub. No.: US 2025/0020675 A1
(43) Pub. Date: Jan. 16, 2025

(54) Application of Lipid Biomarkers in Diagnosis and Early Warning of Fatty Liver in Periparturient Dairy Cows

(71) Applicant: Shandong Agricultural University, Taian (CN)

(72) Inventors: Kerong SHI, Taian (CN); Tingjun LIU, Taian (CN); Xianpeng HOU, Taian (CN); Xiaoran YAN, Taian (CN); Sisi FAN, Taian (CN); Xuan ZHANG, Taian (CN)

(21) Appl. No.: 18/762,649

(22) Filed: Jul. 3, 2024

(30) Foreign Application Priority Data
Jul. 10, 2023 (CN) 202310838281.7

(51) Int. Cl. G01N 33/92

(52) U.S. Cl. CPC G01N 33/92; G01N 2405/02; G01N 2405/06; G01N 2800/085

(57) ABSTRACT

The present application belongs to the technical field of biology. Provided is an application of lipid biomarkers in diagnosis and early warning of fatty liver in periparturient dairy cows. 20 lipid biomarkers found in the present disclosure have diagnostic values in recognizing, early warning, and diagnosing fatty liver diseases in dairy cows at corresponding disease stages. The lipid biomarkers of the present disclosure may be used for identifying and diagnosing the dairy cows with fatty liver.

### CROSS-REFERENCE TO RELATED APPLICATIONS

The present application claims the benefit of the priority of the Chinese patent application with the application No. 202310838281.7, filed to the China National Intellectual Property Administration on Jul. 10, 2023, the entire content of which is incorporated in this application by reference.

### TECHNICAL FIELD

The present disclosure relates to the technical field of biology, and specifically to an application of lipid biomarkers in diagnosis and early warning of fatty liver in periparturient dairy cows.

### BACKGROUND OF THE INVENTION

Fatty liver is a common metabolic disorder disease in periparturient dairy cows. Conventional methods for diagnosing fatty liver in dairy cows include clinical symptoms, liver enzymology tests, ultrasound, liver histopathology tests, etc. However, there is certain limitation in the operability and accuracy of the conventional methods, such that new methods need to be developed to improve the accuracy of diagnosing the fatty liver in the dairy cows.

Fatty liver disease in the periparturient dairy cows is a metabolic disease that accompanies the deposition of lipids in the liver. The lipids are a major component of the fatty liver disease, are an important constituent that tends to accumulate during metabolism, the core of the fatty liver disease, and represent the metabolic disorder of the fatty liver disease in the periparturient dairy cows. The periparturient dairy cows need a lot of energy during milk production, and fatty acids are one of the most key energy sources in metabolism, such that the lipids have an important role in the diagnosis of the fatty liver disease in the periparturient dairy cows.

Lipidomics shows great potential on diagnosing the fatty liver disease in the dairy cows. The lipidomics is a high-throughput technology, which may quantitatively measure the content of lipid molecules in the serum and liver tissue of the periparturient dairy cows and perform identification and analysis on constituents. The lipidomics technology may clearly detect changes in lipid metabolism, and provides valuable information to understand the pathogenesis, early diagnosis, and treatment of the disease.

Lipid biomarkers are biomolecules that are stably present in organisms and measurable with respect to lipid metabolism and functional states; and the lipid biomarkers have great potential in diagnosing the fatty liver disease in the periparturient dairy cows. In recent years, more and more studies indicate that, the lipid biomarkers may serve as a new disease screening tool, so as to recognize and diagnose the fatty liver disease in the periparturient dairy cows earlier.

To sum up, for the fatty liver disease in the periparturient dairy cows, the lipid biomarkers may better understand the onset and follow-up of the disease and the monitoring of treatment effects by monitoring changes in serum levels of lipid metabolites, such that a new idea is provided for early diagnosis and individualized treatment of the fatty liver disease in the periparturient dairy cows.

### SUMMARY OF THE INVENTION

In view of the prior art, the present disclosure is intended to provide an application of lipid biomarkers in diagnosis and early warning of fatty liver in periparturient dairy cows.

In order to implement the above objective, the present disclosure uses the following technical solutions.

A first aspect of the present disclosure provides an application of the following one or more lipids in diagnosis and early warning of fatty liver in periparturient dairy cows as a biomarker:
(1) Ceramide (Cer) (d18:0/22:0);
(2) Diacylglycerol (DAG) (34:1);
(3) ether Phosphatidyl Ethanolamine (ePE) (36:4);
(4) Lysophosphatidylcholine (LPC), selected from LPC(17:0), LPC(20:0), LPC(20:2), or LPC(28:0):
(5) Lysophosphatidylethanolamine (LPE) (20:5);
(6) Phosphatidylcholine (PC), selected from PC(26:0), PC(42:6), PC(O-30:0), or PC(O-32:3):
(7) Phosphatidylethanolamine (PE), selected from PE(34:4), PE(36:2), or PE(36:3); and
(8) Triglyceride (TG), selected from TG(47:0), TG(52:5), TG(52:6), TG(54:6), or TG(56:5).

The 20 lipids as the biomarkers all can accurately recognize the fatty liver in the periparturient dairy cows, and an Area Under Curve (AUC) of each lipid is consistent with diagnostic significance, and has a high clinical diagnostic application value; furthermore, when a plurality of lipids are used in combination, the AUC is closer to 1 than a single lipid is used, such that a diagnostic effect is better.

Further provided is an application of the at least one lipid in development and/or preparation of products for recognizing, early warning, identifying, and diagnosing fatty liver in the periparturient dairy cows as a biomarker.

Further, when different lipids are used singly or in combination, different diagnostic effects may be achieved. Details are as follows.

Four lipids of TG(52:5), TG(56:5), TG(54:6), and TG(52:6), which are used singly or in combination, may be used for diagnosing low-grade fatty liver in the periparturient dairy cows.

Seven lipids of PE(36:3), PE(36:2), PE(34:4), TG(52:5), TG(47:0), PC(26:0), and TG(56:5), which are used singly or in combination, may be used for diagnosing mid-grade fatty liver in the periparturient dairy cows.

Eight lipids of PC(42:6), LPC(17:0), PC(O-32:3), LPC(20:2), LPC(20:0), TG(47:0), PE(34:4), and TG(52:5), which are used singly or in combination, may be used for diagnosing high-grade fatty liver in the periparturient dairy cows.

Four lipids of LPC(20:2), Cer(d18:0/22:0), LPC(20:0), and LPE(20:5), which are used singly or in combination, may be used for distinguishing dairy cows with high-grade fatty liver and dairy cows with mid-grade fatty liver in a periparturient period.

Five lipids of ePE(36:4), LPC(28:0), PC(O-30:0), DAG(34:1), and PC(26:0), which are used singly or in combination, may be used for distinguishing dairy cows with mid-grade fatty liver and dairy cows with low-grade fatty liver in a periparturient period.

The lipids in the following (1)-(2), which are used singly or in combination, may be used for distinguishing dairy cows with fatty liver and healthy dairy cows in a periparturient period:
(1) TG(52:5), PC(42:6), LPC(20:2), TG(54:6), TG(47:0), PE(36:2), or PE(34:4); and
(2) TG(52:5), PE(34:4), TG(47:0), PC(42:6), LPC(20:2), or PC(O-32:3).

Five lipids of TG(47:0), PE(34:4), PC(O-32:3), LPC(28:0), and PC(26:0), which are used singly or in combination, may be used for distinguishing dairy cows with fatty liver and basically-healthy dairy cows in a periparturient period.

Seven lipids of LPC(20:2), LPC(20:0), LPC(17:0), TG(47:0), PC(O-32:3), Cer(d18:0/22:0), and LPE(20:5), which are used singly or in combination, may be used for diagnosing dairy cows with high-grade fatty liver and dairy cows in other conditions in a periparturient period.

A second aspect of the present disclosure provides an application of a reagent for testing at least one lipid in (1)-(8) mentioned above in preparation of products for non-invasive recognition of fatty liver disease in periparturient dairy cows.

Further, the reagent is a reagent for testing lipids in the serum of dairy cows, or a reagent for directly testing lipids in a blood sample.

The present disclosure has the following beneficial effects.

(1) 20 lipid biomarkers found in the present disclosure have diagnostic values in recognizing, early warning, and diagnosing fatty liver diseases in dairy cows at corresponding disease stages. The lipid biomarkers of the present disclosure may be used for identifying and diagnosing the dairy cows with fatty liver, and have high specificity and sensitivity; the markers are stable in nature, simple in operation, low in price, and are a non-invasive detection means; and the markers have no harm or injury to dairy cows, which are in line with the concept of animal welfare and healthy farming, and facilitates the rapid grasping of the conditions of the periparturient dairy cows with fatty liver by breeding personnel, such that individualized treatment and assistance plans are timely formulated for breeding production.

(2) In conjunction with the screening and verification of 37 periparturient dairy cows, it was found that the lipid biomarkers provided in the present disclosure have high sensitivity and specificity on recognition and diagnosis of the fatty liver, and has significant use value and significance for future diagnosis of the fatty liver in the periparturient dairy cows.

### BRIEF DESCRIPTION OF DRAWINGS

FIG. 1 is a discovery flowchart of a biomarker according to the present disclosure.

FIG. 2 shows fat content of liver tissue of each group of an experimental dairy cow population.

FIG. 3A-FIG. 3C show volcano plots of multidimensional metabolites of each group.

FIG. 4A-FIG. 4C show volcano plots of unidimensional metabolites of each group.

FIG. 5A-FIG. 5B show Venn diagrams of each group, where A represents the expression of 44 lipids in five groups during screening, and B represents the expression of final 20 lipids in five groups.

FIG. 6 is a Venn diagram of the distribution of lipids with higher diagnostic power in 4 combined comparison groups.

### DETAILED DESCRIPTION OF THE INVENTION

It should be noted that, the following detailed description is exemplary and intended to provide further description of the present application. Unless otherwise defined, all technical and scientific terms used herein have the same meaning as commonly understood by those of ordinary skill in the art to which the present application belongs.

As described above, lipids may be used as more effective biomarkers for the diagnosis and prognosis of many diseases. Based on this, the present disclosure is intended to use a bioinformatics method to screen, in lipids of the serum of the periparturient dairy cows, lipid biomarkers having recognition, early warning, identification, and diagnostic values, and candidate lipid biomarkers are evaluated and verified to solve the problem that has been plaguing the field with difficult recognition, early warning, and diagnosis of fatty liver in the periparturient dairy cows.

In order to enable those skilled in the art to understand the technical solutions of the present application more clearly, the technical solutions of the present application will be described in detail with reference to specific embodiments, and an invention flow is shown in FIG. 1.

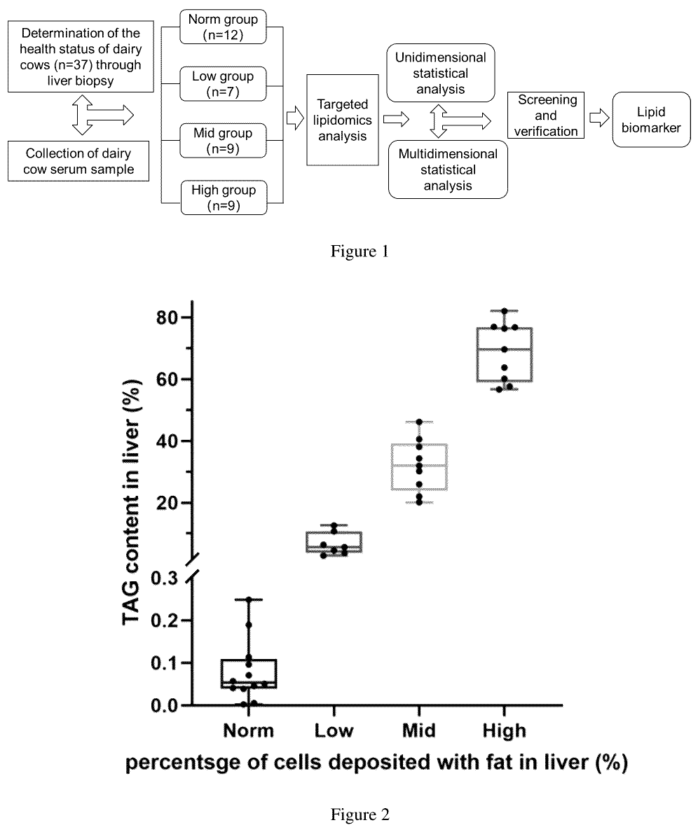

Holstein cows with less than 3 parities after 7±2 days postpartum (n=37) are selected for liver biopsy, and the serum of each cow under a health state at fasting is acquired. According to tissue oil red O staining results, and the percentage of liver cells containing lipid droplets, dairy cow samples are divided into 4 groups (n=37): a normal group (Norm group), fat content=0.080%±0.073%, and n=12; a low-grade fatty liver group (Low group), fat content=6.614%±3.662%, n=7; a mid-grade fatty liver group (Mid group), fat content=32.143%±8.639%, n=9; and a high-grade fatty liver group (High group), fat content=68.896%±9.603%, n=9. The fat content of liver tissue of each group of a dairy cow population is shown in FIG. 2.

Further, targeted lipidomics analysis is performed on the serum of the periparturient dairy cow population that has been subjected to liver biopsy identification with an Ultra-high Performance Liquid Chromatography Triple Quadrupole Mass Spectrometer (UPLC-TQMS), so as to obtain all lipid expression profiles.

Further, there are total 8 comparison groups, which are Low_vs_Norm, Mid_vs_Norm, High_vs_Norm, High_vs_Mid, Mid_vs_Low, High_vs_Mid_vs_Norm, Mid_vs_Low_vs_Norm, and High_vs_Mid_vs_Low_vs_Norm, respectively.

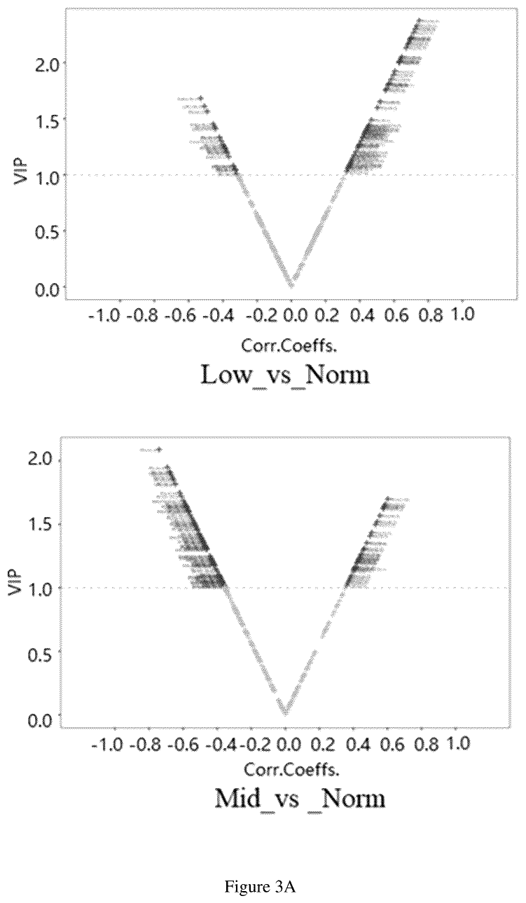

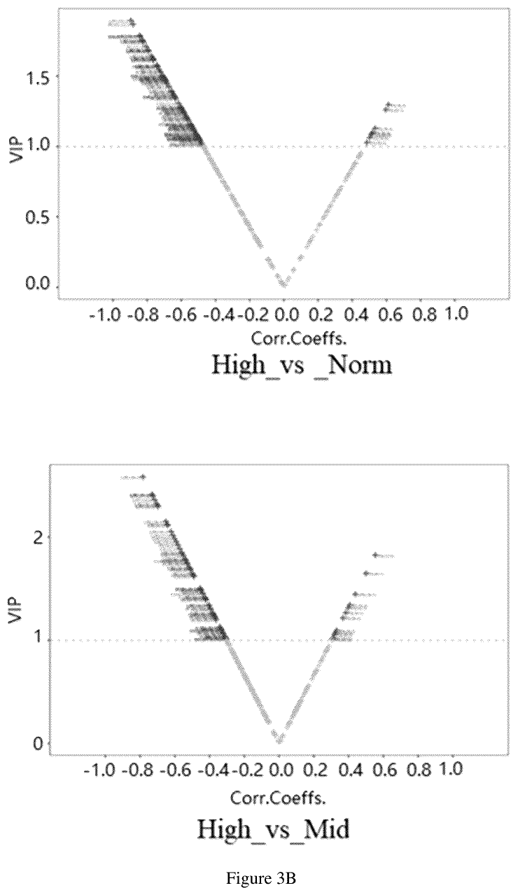

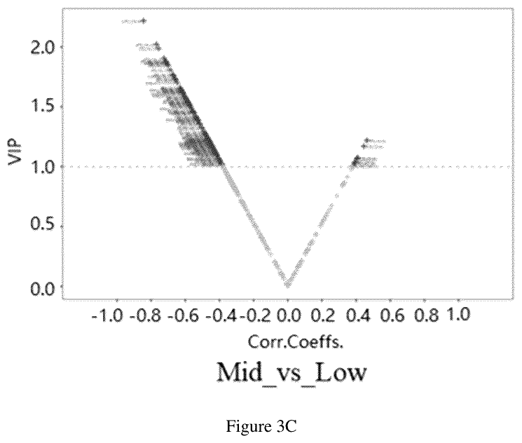

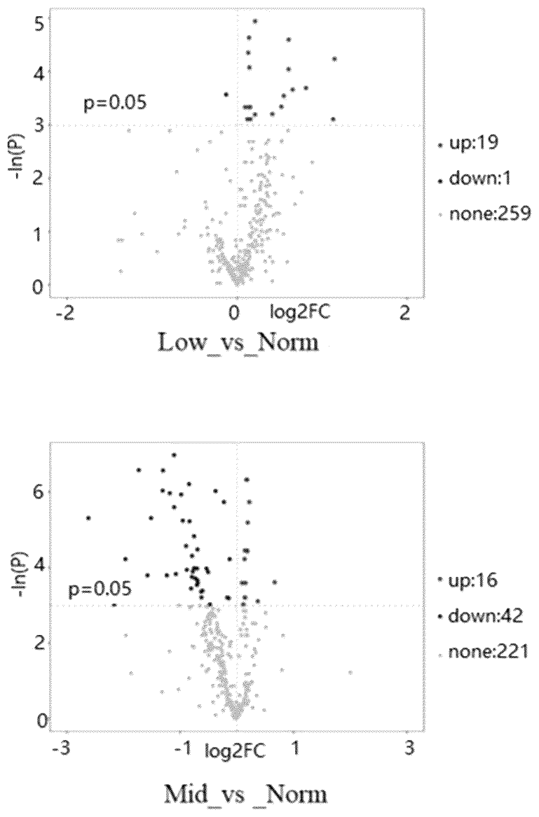

Further, dimension reduction processing is performed on each comparison group in data with a multidimensional statistical Principal Component Analysis (PCA) model, a Partial Least Squares Discriminant Analysis (PLS-DA), and an Orthogonal Partial Least Squares Discriminant Analysis (OPLS-DA). On the basis of an (OPLS-DA) model result, a volcano plot is configured to screen reliable lipid markers; and the volcano plot comprehensively investigates the contribution (Variable Importance in Projection (VIP) value) of lipids to model groups and reliability (Corr. Coeffs, which is a correlation coefficient between a metabolite and a first principal component) of metabolites. The volcano plots of multidimensional metabolites are shown in FIG. 3A-FIG. 3C, and lipids with VIP>1 are selected.

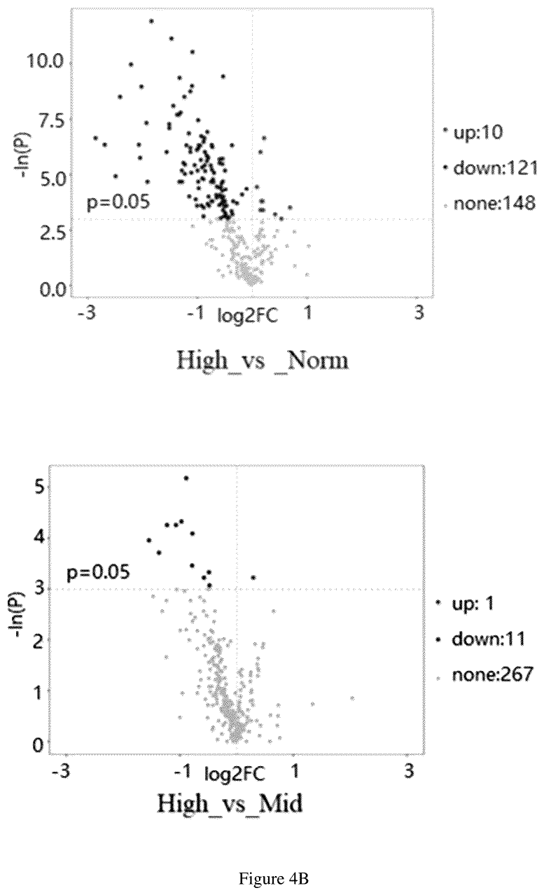

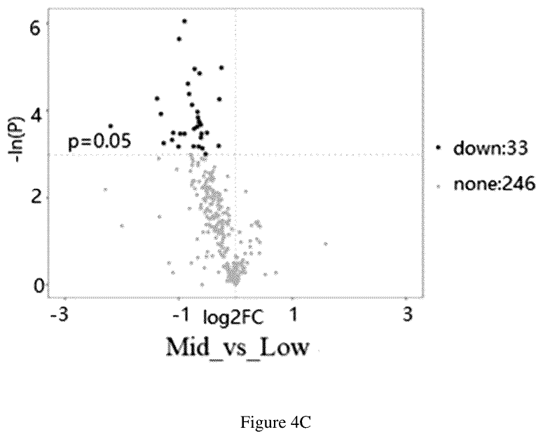

Further, differential metabolites between two groups are obtained by means of a unidimensional test (a T Test or a Mann-Whitney U Test is selected according to the normality and homogeneity of variance of the data), especially when a robust discrimination model cannot be established by means of multidimensional statistics (for example, the distribution of sample categories between groups is uneven or deviation within the group is excessive). The volcano plots of unidimensional metabolites are shown in FIG. 4A-FIG. 4C. The volcano plots show differential metabolites that are screened on the basis of unidimensional statistical analysis. In the current analysis, unidimensional statistical analysis screening is labeled as P<0.05, |log 2(FC)|≥0 (Fold Change (FC)).

Further, the differential lipids commonly acquired in unidimensional and multidimensional manners in each foregoing comparison group are screened; and on the basis of unidimensional and multidimensional analysis, potential lipid biomarkers (comparison is performed among a plurality of groups, OPLS-DA analysis cannot be performed, and results are results using unidimensional differential lipids) possibly having biological values are selected. Multidimensional and unidimensional common results meet double standard screening, and are the most reliable differential metabolites, such that the results are more likely to be potential biomarkers. A total of 130 (duplicates removed) differential lipids are obtained from the comparison group results.

Further, for the obtained lipids differentially expressed among the groups, the diagnostic values of the lipids obtained among the comparison groups are evaluated by means of Receiver Operating Characteristic (ROC) curve screening analysis. When the AUC of an ROC curve is between 0.7 and 0.9 (simultaneously P<0.05), it indicates that the AUC is moderately accurate. When the AUC of the ROC curve is greater than 0.9 (simultaneously P<0.05), it indicates that the AUC is highly accurate.

Further, screening standards of the comparison groups include: the screening standard of a Low_vs_Norm comparison group being AUC>0.83, P<0.05; the screening standard of a Mid_vs_Norm comparison group being AUC>0.85, P<0.05; the screening standard of a High_vs_Norm comparison group being that up-regulated lipids are selected with AUC>0.85, P<0.05, and down-regulated lipids are selected with AUC>0.9, P<0.05; the screening standard of a High_vs_Mid comparison group being AUC>0.83, P<0.05; and the screening standard of a Mid_vs_Low comparison group being AUC>0.85, P<0.05.

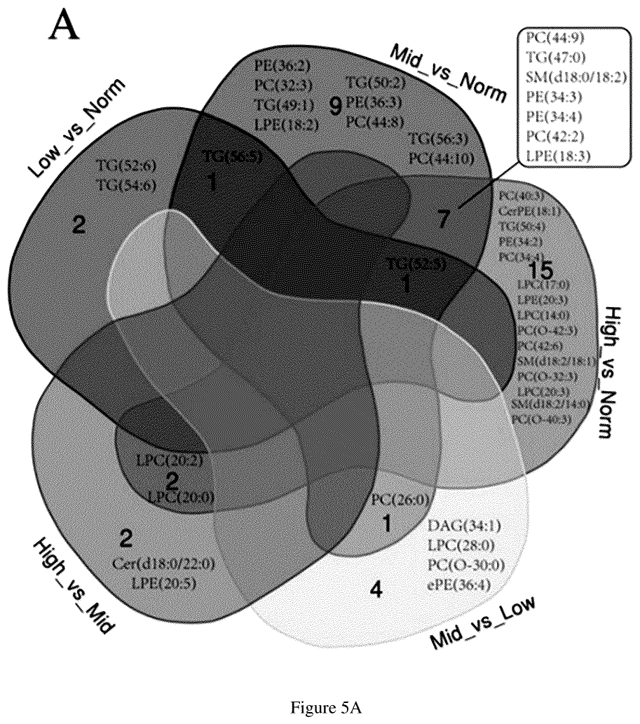

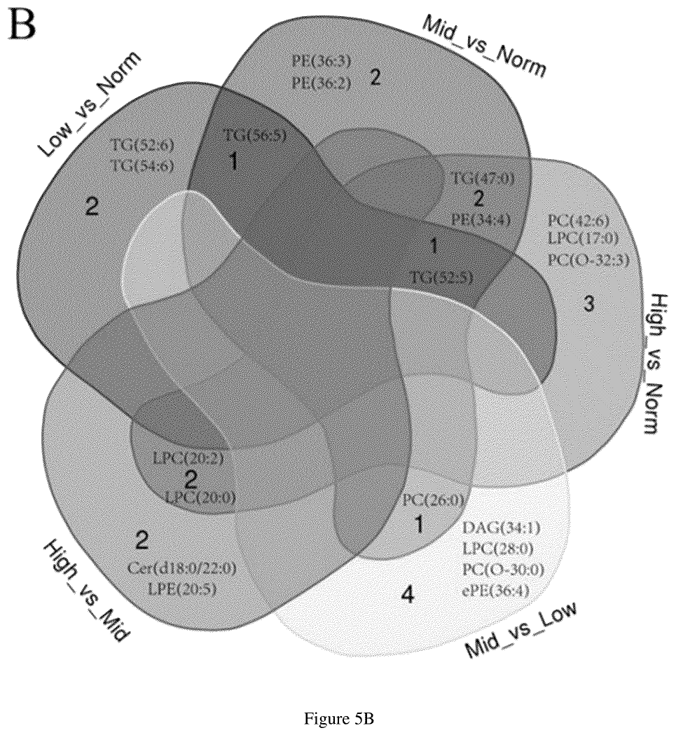

Further, under the above standards, a total of 47 (duplicates removed) lipids having diagnostic values are obtained from the 5 comparison groups; ROC analysis results of the Mid_vs_Norm, High_vs_Norm, and High_vs_Mid comparison groups under the 47 lipid results are compared with the differential lipids obtained from the High_vs_Mid_vs_Norm comparison group, so as to obtain differential lipids shared by the 4 groups; ROC analysis results of the Low_vs_Norm, Mid_vs_Norm, and Mid_vs_Low comparison groups under the 47 lipid results are compared with the differential lipids obtained from the Mid_vs_Low_vs_Norm comparison group, so as to obtain differential lipids shared by the 4 groups; and ROC analysis results of the Low_vs_Norm, Mid_vs_Norm, High_vs_Norm, High_vs_Mid, and Mid_vs_Low comparison groups under the 47 lipid results are compared with the differential lipids obtained from the High_vs_Mid_vs_Low_vs_Norm comparison group, so as to obtain differential lipids shared by the 6 groups. A total of 44 (duplicates removed) candidate lipid biomarkers having diagnostic values in the 8 comparison groups are obtained, referring to the Venn diagram in FIG. 5A and Table 1.

Further, due to too many candidate lipid biomarkers obtained, the lipids (9) that only appear in the Mid_vs_Norm comparison group are simplified, and the lipids with AUC>0.9 and P<0.05 are selected; the lipids (15) that only appear in the High_vs_Norm comparison group are simplified, and the lipids with AUC>0.98 are selected; the lipids (7) that only appear in the Mid_vs_Norm and High_vs_Norm comparison groups are simplified, and the lipids with AUC>0.98 are selected; and the remaining differential lipids are unchanged. Therefore, 20 lipid biomarkers having diagnostic values are obtained, and may be used as non-invasive lipid biomarkers for recognizing dairy cows with fatty liver disease at corresponding disease severities, respectively being Cer(d18:0/22:0), DAG(34:1), ePE(36:4), LPC(17:0), LPC(20:0), LPC(20:2), LPC(28:0), LPE(20:5), PC(26:0), PC(42:6), PC(O-30:0), PC(O-32:3), PE(34:4), PE(36:2), PE(36:3), TG(47:0), TG(52:5), TG(52:6), TG(54:6), and TG(56:5), referring to the Venn diagram in FIG. 5B and Table 2 (conjoint analysis is performed on the corresponding lipids obtained from each comparison group).

Further, 20 lipid biomarkers having diagnostic values are obtained, and the situation of the lipids obtained between the corresponding comparison groups is shown below.

The screening standard of the Low_vs_Norm comparison group is AUC>0.83, P<0.05, and 4 lipid biomarkers, which are TG(52:5), TG(56:5), TG(54:6), and TG(52:6), are obtained.

The screening standard of the Mid_vs_Norm comparison group is AUC>0.85, P<0.05, and 7 lipid biomarkers, which are PE(36:3), PE(36:2), PE(34:4), TG(52:5), TG(47:0), PC(26:0), and TG(56:5), are obtained.

The screening standard of the High_vs_Norm comparison group is that up-regulated lipids are selected with AUC>0.85, P<0.05, and down-regulated lipids are selected with AUC>0.9, P<0.05, and 8 lipid biomarkers, which are PC(42:6), LPC(17:0), PC(O-32:3), LPC(20:2), LPC(20:0), TG(47:0), PE(34:4), and TG(52:5), are obtained.

The screening standard of the High_vs_Mid comparison group is AUC>0.83, P<0.05, and 4 lipid biomarkers, which are LPC(20:2), Cer(d18:0/22:0), LPC(20:0), and LPE(20:5), are obtained.

The screening standard of the Mid_vs_Low comparison group is AUC>0.85, P<0.05, and 5 lipid biomarkers, which are ePE(36:4), LPC(28:0), PC(O-30:0), DAG(34:1), and PC(26:0), are obtained.

Further, the 20 lipid biomarkers obtained above are diagnosed in a combined group.

Further, the High group, the Mid group, and the Low group are used as disease groups, which are recorded as HML groups; and the diagnostic values of the obtained lipids are evaluated by performing ROC analysis on an HML_vs_Norm comparison group. The selection standard is AUC>0.85, P<0.05, among 20 lipids obtained, 7 lipids have higher diagnostic power, and thus may be used as biomarkers, which respectively are TG(52:5), PC(42:6), LPC(20:2), TG(54:6), TG(47:0), PE(36:2), and PE(34:4).

Further, the High group and the Mid group are used as disease groups, which are recorded as HM groups; and the diagnostic values of the obtained lipids are evaluated by performing ROC analysis on an HM_vs_Norm comparison group. The selection standard is AUC>0.89, P<0.05, among 20 lipids obtained, 6 lipids have higher diagnostic power, and thus may be used as biomarkers, which respectively are TG(52:5), PE(34:4), TG(47:0), PC(42:6), LPC(20:2), and PC(O-32:3).

Further, according to laboratory research experience, low-grade fatty liver may be restored to a normal state by means of feeding adjustment, such that the High group and the Mid group are used as the disease groups, which are recorded as the HM groups; the Low group and the Norm group are used as basically-normal groups, which are recorded as LN groups; and the diagnostic values of the obtained lipids are evaluated by performing ROC analysis on an HM_vs_LN comparison group. The selection standard is AUC>0.85, P<0.05, among 20 lipids obtained, 5 lipids have higher diagnostic power, and thus may be used as biomarkers, which respectively are TG(47:0), PE(34:4), PC(O-32:3), LPC(28:0), and PC(26:0).

Further, in order to distinguish the High group and other states, the Mid group, the Low group, and the Norm group are combined as one group, which is recorded as an MLN group; and the diagnostic values of the obtained lipid biomarkers are evaluated by performing ROC curve screening analysis on a High_vs_MLN comparison group. The selection standard is AUC>0.85, P<0.05, among 20 lipids obtained, 7 lipids have higher diagnostic power, and thus may be used as biomarkers, which respectively are LPC(20:2), LPC(20:0), LPC(17:0), TG(47:0), PC(O-32:3), Cer(d18:0/22:0), and LPE(20:5).

In order to enable those skilled in the art to understand the technical solutions of the present application more clearly, the technical solutions of the present application will be described in detail with reference to specific embodiments.

Test materials used in the embodiments of the present disclosure are conventional test materials in the art and are commercially available.

### Embodiment 1: Biological Sample Situations and Methods Involved in the Present Disclosure

According to results of fat content of the collected liver tissue, the degree of fatty liver in dairy cows was classified (referring to FIG. 2 for diagnosis situations); and according to different fat content of diseased dairy cows, all test samples were divided into 4 groups (n=37): a normal group (Norm group), fat content=0.080%±0.073%, and n=12; a low-grade fatty liver group (Low group), fat content=6.614%±3.662%, n=7; a mid-grade fatty liver group (Mid group), fat content=32.143%±8.639%, n=9; and a high-grade fatty liver group (High group), fat content=68.896%±9.603%, n=9. The fat content of liver tissue of each group of a dairy cow population was shown in FIG. 2.

Targeted lipidomics analysis was performed on the serum of the periparturient dairy cow population that had been subjected to liver biopsy identification by using an UPLC-TQMS, so as to obtain all lipid expression profiles, and a total of 279 lipids were identified.

### Embodiment 2: Multidimensional Statistical Analysis Screening

Dimension reduction processing was performed on each comparison group in data with a multidimensional statistical PCA model, a PLS-DA, and an OPLS-DA. On the basis of an (OPLS-DA) model result, a volcano plot was configured to screen reliable lipid markers; and the volcano plot comprehensively investigated the contribution (VIP value) of lipids to model groups and reliability (Corr. Coeffs, which was a correlation coefficient between a metabolite and a first principal component) of metabolites. The volcano plots of multidimensional metabolites were shown in FIG. 3A-FIG. 3C, and lipids with VIP>1 were selected.

### Embodiment 3: Unidimensional Statistical Analysis Screening

Differential metabolites between two groups were obtained by means of a unidimensional test (a T Test or a Mann-Whitney U Test was selected according to the normality and homogeneity of variance of the data), especially when a robust discrimination model was unable to be established by means of multidimensional statistics (for example, the distribution of sample categories between groups was uneven or deviation within the group was excessive). The volcano plots of unidimensional metabolites were shown in FIG. 4A-FIG. 4C. The volcano plots showed differential metabolites that were screened on the basis of unidimensional statistical analysis. In the current analysis, unidimensional statistical analysis screening was labeled as P<0.05, |log 2(FC)|≥0.

### Embodiment 4: Common Result Obtained by Means of Multidimensional and Unidimensional Double Standards

A common result obtained by means of multidimensional and unidimensional double standards of a Low_vs_Norm comparison group was 20 lipids.

A common result obtained by means of multidimensional and unidimensional double standards of a Mid_vs_Norm comparison group was 56 lipids.

A common result obtained by means of multidimensional and unidimensional double standards of a High_vs_Norm comparison group was 106 lipids.

A common result obtained by means of multidimensional and unidimensional double standards of a High_vs_Mid comparison group was 12 lipids.

A common result obtained by means of multidimensional and unidimensional double standards of a Mid_vs_Low comparison group was 32 lipids.

A total of 107 lipids were obtained from a High_vs_Mid_vs_Norm comparison group (OPLS-DA analysis was unable to be performed due to comparison among a plurality of groups, such that a potential biomarker result was the same as a unidimensional differential metabolite result).

A total of 46 lipids were obtained from a Mid_vs_Low_vs_Norm comparison group (OPLS-DA analysis was unable to be performed due to comparison among the plurality of groups, such that the potential biomarker result was the same as the unidimensional differential metabolite result).

A total of 107 lipids were obtained from an High_vs_Mid_vs_Low_vs_Norm comparison group (OPLS-DA analysis was unable to be performed due to comparison among the plurality of groups, such that the potential biomarker result was the same as the unidimensional differential metabolite result), and there were a total of 8 comparison groups described above.

### Embodiment 5: Determination of Final 20 Lipid Biomarkers

Further, screening standards of the comparison groups included: the screening standard of the Low_vs_Norm comparison group being AUC>0.83, P<0.05; the screening standard of the Mid_vs_Norm comparison group being AUC>0.85, P<0.05; the screening standard of the High_vs_Norm comparison group being that up-regulated lipids were selected with AUC>0.85, P<0.05, and down-regulated lipids were selected with AUC>0.9, P<0.05; the screening standard of the High_vs_Mid comparison group being AUC>0.83, P<0.05; and the screening standard of the Mid_vs_Low comparison group being AUC>0.85, P<0.05.

Further, under the above standards, a total of 47 (duplicates removed) lipids having diagnostic values were obtained from the 5 comparison groups of Low_vs_Norm, Mid_vs_Norm, High_vs_Norm, High_vs_Mid, and Mid_vs_Low; ROC analysis results of the Mid_vs_Norm, High_vs_Norm, and High_vs_Mid comparison groups under the 47 lipid results were compared with the differential lipids obtained from the High_vs_Mid_vs_Norm comparison group, so as to obtain differential lipids shared by the 4 groups; ROC analysis results of the Low_vs_Norm, Mid_vs_Norm, and Mid_vs_Low comparison groups under the 47 lipid results were compared with the differential lipids obtained from the Mid_vs_Low_vs_Norm comparison group, so as to obtain differential lipids shared by the 4 groups; and ROC analysis results of the Low_vs_Norm, Mid_vs_Norm, High_vs_Norm, High_vs_Mid, and Mid_vs_Low comparison groups under the 47 lipid results were compared with the differential lipids obtained from the High_vs_Mid_vs_Low_vs_Norm comparison group, so as to obtain differential lipids shared by the 5 groups. A total of 44 (duplicates removed) candidate lipid biomarkers having diagnostic values in the 8 comparison groups were obtained, referring to Table 1 and the Venn diagram in FIG. 5A.

TABLE 1
A total of 44 candidate lipid biomarkers having diagnostic values in the
8 comparison groups were obtained through preliminary screening.

| Lipid | HMDB database No. | KEGG database No. | Class |
|---|---|---|---|
| Cer(d18:0/22:0) | HMDB0011765 | C00195 | Cer |
| CerPE(18:1) | NA | C06062 | CerPE |
| DAG(34:1) | HMDB0007101 | C00641 | DAG |
| ePE(36:4) | NA | C04475 | PE |
| LPC(14:0) | HMDB0010379 | C04230 | LPC |
| LPC(17:0) | HMDB0012108 | C04230 | LPC |
| LPC(20:0) | HMDB0010390 | C04230 | LPC |
| LPC(20:2) | HMDB0010392 | C04230 | LPC |
| LPC(20:3) | HMDB0010393 | C04230 | LPC |
| LPC(28:0) | NA | C04230 | LPC |
| LPE(18:2) | HMDB0011507 | C04438 | LPE |
| LPE(18:3) | HMDB0011508 | C04438 | LPE |
| LPE(20:3) | HMDB0011515 | C04438 | LPE |
| LPE(20:5) | HMDB0011519 | C04438 | LPE |
| PC(26:0) | NA | C00157 | PC |
| PC(32:3) | HMDB0007907 | C00157 | PC |
| PC(34:4) | HMDB0008007 | C00157 | PC |
| PC(40:3) | HMDB0008216 | C00157 | PC |
| PC(42:2) | HMDB0008157 | C00157 | PC |
| PC(42:6) | HMDB0008576 | C00157 | PC |
| PC(44:10) | HMDB0008713 | C00157 | PC |
| PC(44:8) | HMDB0008616 | C00157 | PC |
| PC(44:9) | HMDB0008647 | C00157 | PC |
| PC(O-30:0) | HMDB0013341 | C00157 | PC |
| PC(O-32:3) | NA | C05212 | PC |
| PC(O-40:3) | HMDB0013445 | C00157 | PC |
| PC(O-42:3) | HMDB0013458 | C00157 | PC |
| PE(34:2) | HMDB0008928 | C00350 | PE |
| PE(34:3) | HMDB0008961 | C00350 | PE |
| PE(34:4) | HMDB0008962 | C00350 | PE |
| PE(36:2) | HMDB0008934 | C00350 | PE |
| PE(36:3) | HMDB0008935 | C00350 | PE |
| SM(d18:0/18:2) | NA | C00550 | SM |
| SM(d18:2/14:0) | HMDB0240637 | C00550 | SM |
| SM(d18:2/18:1) | NA | C00550 | SM |
| TG(47:0) | HMDB0071688 | C00422 | TG |
| TG(49:1) | HMDB0045506 | C00422 | TG |
| TG(50:2) | HMDB0005362 | C00422 | TG |
| TG(50:4) | HMDB0047962 | C00422 | TG |
| TG(52:5) | HMDB0005380 | C00422 | TG |
| TG(52:6) | HMDB0042646 | C00422 | TG |
| TG(54:6) | HMDB0048747 | C00422 | TG |
| TG(56:3) | HMDB0042229 | C00422 | TG |
| TG(56:5) | HMDB0044321 | C00422 | TG |

Note: NA indicated that the number of the lipid was not available in the database.

Further, due to too many candidate lipid biomarkers obtained, the lipids (9) that only appeared in the Mid_vs_Norm comparison group were simplified, and the lipids with AUC>0.9 and P<0.05 were selected; the lipids (15) that only appeared in the High_vs_Norm comparison group were simplified, and the lipids with AUC>0.98 were selected; the lipids (7) that only appeared in the Mid_vs_Norm and High_vs_Norm comparison groups were simplified, and the lipids with AUC>0.98 were selected; and the remaining differential lipids were unchanged. Therefore, 20 lipid biomarkers having diagnostic values were obtained, and might be used as non-invasive lipid biomarkers for recognizing dairy cows with fatty liver disease at corresponding disease severities, referring to Table 2 and the Venn diagram (FIG. 5B).

TABLE 2
A total of 20 candidate lipid biomarkers having diagnostic values in
the 8 comparison groups were obtained through final screening.

| Lipid | HMDB database No. | KEGG database No. | Class |
|---|---|---|---|
| Cer(d18:0/22:0) | HMDB0011765 | C00195 | Cer |
| DAG(34:1) | HMDB0007101 | C00641 | DAG |
| ePE(36:4) | NA | C04475 | PE |
| LPC(17:0) | HMDB0012108 | C04230 | LPC |
| LPC(20:0) | HMDB0010390 | C04230 | LPC |
| LPC(20:2) | HMDB0010392 | C04230 | LPC |
| LPC(28:0) | NA | C04230 | LPC |
| LPE(20:5) | HMDB0011519 | C04438 | LPE |
| PC(26:0) | NA | C00157 | PC |
| PC(42:6) | HMDB0008576 | C00157 | PC |
| PC(O-30:0) | HMDB0013341 | C00157 | PC |
| PC(O-32:3) | NA | C05212 | PC |
| PE(34:4) | HMDB0008962 | C00350 | PE |
| PE(36:2) | HMDB0008934 | C00350 | PE |
| PE(36:3) | HMDB0008935 | C00350 | PE |
| TG(47:0) | HMDB0071688 | C00422 | TG |
| TG(52:5) | HMDB0005380 | C00422 | TG |
| TG(52:6) | HMDB0042646 | C00422 | TG |
| TG(54:6) | HMDB0048747 | C00422 | TG |
| TG(56:5) | HMDB0044321 | C00422 | TG |

Note: NA indicated that the number of the lipid was not available in the database.

### Embodiment 6: Diagnostic Power Analysis and Conjoint Analysis (ROC Analysis) of Lipid Biomarkers Obtained from Each Group

Further, 20 lipid biomarkers having diagnostic values were obtained, and the lipids were obtained between the corresponding comparison groups; and with SPSS data statistics software, a combined marker variable combination was determined with a binary logistic regression analysis method, and the situation of performing conjoint analysis on the corresponding lipids obtained between the corresponding comparison groups was shown below.

The screening standard of the Low_vs_Norm comparison group was AUC>0.83, P<0.05, and 4 lipid biomarkers were obtained, referring to Table 3.

TABLE 3
ROC analysis and combined diagnosis of 4 lipid biomarkers
obtained from Low_vs_Norm comparison group.

| | | SEM | | | | | Best cutoff | |
|---|---|---|---|---|---|---|---|---|
| Lipid | AUC | (±) | P-value | 95% CI | Sensitivity | Specificity | value | Trend |
| TG(52:5) | 0.869 | 0.086 | 0.009 | 0.700~1 | 0.857 | 0.833 | 0.69 | Up |
| TG(56:5) | 0.857 | 0.098 | 0.011 | 0.665~1 | 0.857 | 0.833 | 0.69 | Up |
| TG(54:6) | 0.845 | 0.100 | 0.014 | 0.649~1 | 0.714 | 0.917 | 0.631 | Up |
| TG(52:6) | 0.833 | 0.108 | 0.018 | 0.622~1 | 0.857 | 0.750 | 0.607 | Up |
| Top2 combined | 0.881 | 0.088 | 0.007 | 0.708~1 | 0.857 | 0.917 | 0.774 | — |
| diagnosis | | | | | | | | |

The conjoint analysis of the top 2 lipid biomarkers obtained from the Low_vs_Norm comparison group was shown in Table 3; and through analysis, the top 2 lipids were optimal in combined diagnosis effect.

The screening standard of the Mid_vs_Norm comparison group was AUC>0.85, P<0.05, and 7 lipid biomarkers were obtained, referring to Table 4.

TABLE 4
ROC analysis and combined diagnosis of 7 lipid biomarkers
obtained from Mid_vs_Norm comparison group.

| | | SEM | | | | | Best cutoff | |
|---|---|---|---|---|---|---|---|---|
| Lipid | AUC | (±) | P-value | 95% CI | Sensitivity | Specificity | value | Trend |
| PE(36:3) | 0.935 | 0.052 | 0.001 | 0.832~1 | 1.000 | 0.833 | 0.833 | Down |
| PE(36:2) | 0.907 | 0.064 | 0.002 | 0.783~1 | 1.000 | 0.750 | 0.750 | Down |
| PE(34:4) | 0.898 | 0.070 | 0.002 | 0.760~1 | 0.778 | 0.917 | 0.694 | Down |
| TG(52:5) | 0.889 | 0.075 | 0.003 | 0.743~1 | 0.889 | 0.833 | 0.722 | Up |
| TG(47:0) | 0.880 | 0.077 | 0.004 | 0.729~1 | 0.667 | 1.000 | 0.667 | Down |
| PC(26:0) | 0.870 | 0.083 | 0.004 | 0.707~1 | 0.889 | 0.833 | 0.722 | Down |
| TG(56:5) | 0.870 | 0.082 | 0.004 | 0.711~1 | 0.778 | 0.917 | 0.694 | Up |
| Top7 combined | 1.000 | 0 | 0 | 1.000 | 1.000 | 1.000 | 1.000 | — |
| diagnosis | | | | | | | | |

The conjoint analysis of the 7 lipid biomarkers obtained from the Mid_vs_Norm comparison group was shown in Table 4.

The screening standard of the High_vs_Norm comparison group was that up-regulated lipids were selected with AUC>0.85, P<0.05, and down-regulated lipids were selected with AUC>0.9, P<0.05, and 8 lipid biomarkers were obtained, referring to Table 5.

TABLE 5
ROC analysis and combined diagnosis of 8 lipid biomarkers
obtained from High_vs_Norm comparison group.

| | | SEM | | | | | Best cutoff | |
|---|---|---|---|---|---|---|---|---|
| Lipid | AUC | (±) | P-value | 95% CI | Sensitivity | Specificity | value | Trend |
| PC(42:6) | 1.000 | 0 | 0 | 1.000 | 1.000 | 1.000 | 1.000 | Down |
| LPC(17:0) | 0.981 | 0.024 | 0 | 0.934~1 | 0.889 | 1.000 | 0.889 | Down |
| PC(O-32:3) | 0.981 | 0.024 | 0 | 0.935~1 | 1.000 | 0.917 | 0.917 | Down |
| LPC(20:2) | 0.972 | 0.03 | 0 | 0.914~1 | 1.000 | 0.833 | 0.833 | Down |
| LPC(20:0) | 0.963 | 0.036 | 0 | 0.892~1 | 1.000 | 0.833 | 0.833 | Down |
| TG(47:0) | 0.963 | 0.036 | 0 | 0.892~1 | 1.000 | 0.833 | 0.833 | Down |
| PE(34:4) | 0.954 | 0.043 | 0 | 0.869~1 | 1.000 | 0.833 | 0.833 | Down |
| TG(52:5) | 0.898 | 0.07 | 0.002 | 0.761~1 | 0.889 | 0.833 | 0.722 | Up |
| Top 2-8 combined | 1.000 | 0 | 0 | 1.000 | 1.000 | 1.000 | 1.000 | — |
| diagnosis | | | | | | | | |

The conjoint analysis of the Top 2-8 lipid biomarkers obtained from the High_vs_Norm comparison group was shown in Table 5. Since an AUC value of the lipid PC (42:6) in the group had been reached 1, conjoint analysis was performed on the Top 2-8 lipid biomarkers.

The screening standard of the High_vs_Mid comparison group was AUC>0.83, P<0.05, and 4 lipid biomarkers were obtained, referring to Table 6.

TABLE 6
ROC analysis and combined diagnosis of 4 lipid biomarkers
obtained from High_vs_Mid comparison group.

| | | SEM | | | | | Best cutoff | |
|---|---|---|---|---|---|---|---|---|
| Lipid | AUC | (±) | P-value | 95% CI | Sensitivity | Specificity | value | Trend |
| LPC(20:2) | 0.877 | 0.088 | 0.007 | 0.703~1 | 0.778 | 1.000 | 0.778 | Down |
| Cer(d18:0/22:0) | 0.840 | 0.103 | 0.015 | 0.637~1 | 0.667 | 1.000 | 0.667 | Down |
| LPC(20:0) | 0.840 | 0.099 | 0.015 | 0.645~1 | 0.889 | 0.778 | 0.667 | Down |
| LPE(20:5) | 0.833 | 0.097 | 0.017 | 0.644~1 | 0.556 | 1.000 | 0.556 | Down |
| Top4 combined | 1.000 | 0 | 0 | 1.000 | 1.000 | 1.000 | 1.000 | — |
| diagnosis | | | | | | | | |

The conjoint analysis of the 4 lipid biomarkers obtained from the High_vs_Mid comparison group was shown in Table 6.

As shown in Table 7, the screening standard of the Mid_vs_Low comparison group was AUC>0.85, P<0.05, and 5 lipid biomarkers were obtained, referring to Table 7.

TABLE 7
ROC analysis and combined diagnosis of 5 lipid biomarkers
obtained from Mid_vs_Low comparison group.

| | | SEM | | | | | Best cutoff | |
|---|---|---|---|---|---|---|---|---|
| Lipid | AUC | (±) | P-value | 95% CI | Sensitivity | Specificity | value | Trend |
| ePE(36:4) | 0.905 | 0.093 | 0.007 | 0.722~1 | 1.000 | 0.857 | 0.857 | Down |
| LPC(28:0) | 0.905 | 0.077 | 0.007 | 0.754~1 | 0.778 | 1.000 | 0.778 | Down |
| PC(O-30:0) | 0.889 | 0.082 | 0.010 | 0.728~1 | 0.667 | 1.000 | 0.667 | Down |
| DAG(34:1) | 0.857 | 0.096 | 0.017 | 0.669~1 | 0.778 | 0.857 | 0.635 | Down |
| PC(26:0) | 0.857 | 0.100 | 0.017 | 0.660~1 | 0.667 | 1.000 | 0.667 | Down |
| Top5 combined | 1.000 | 0 | 0 | 1.000 | 1.000 | 1.000 | 1.000 | — |
| diagnosis | | | | | | | | |

The conjoint analysis of the 5 lipid biomarkers obtained from the Mid_vs_Low comparison group was shown in Table 7.

Further, the 20 lipid biomarkers obtained above were diagnosed in a combined group.

Further, the High group, the Mid group, and the Low group were used as disease groups, which were recorded as HML groups; and the diagnostic values of the obtained lipids were evaluated by performing ROC analysis on an HML_vs_Norm comparison group. The selection standard was AUC>0.85, P<0.05, among 20 lipids obtained, 7 lipids had higher diagnostic power, and thus might be used as biomarkers, referring to Table 8.

TABLE 8
ROC analysis and combined diagnosis of 7 lipid biomarkers
obtained from HML_vs_Norm comparison group.

| | | SEM | | | | | Best cutoff | |
|---|---|---|---|---|---|---|---|---|
| Lipid | AUC | (±) | P-value | 95% CI | Sensitivity | Specificity | value | Trend |
| TG(52:5) | 0.887 | 0.062 | 0 | 0.765~1.000 | 0.88 | 0.833 | 0.713 | Up |
| PC(42:6) | 0.870 | 0.059 | 0 | 0.754~0.986 | 0.76 | 1.000 | 0.760 | Down |
| LPC(20:2) | 0.863 | 0.060 | 0 | 0.745~0.981 | 0.84 | 0.833 | 0.673 | Down |
| TG(54:6) | 0.820 | 0.068 | 0.002 | 0.686~0.954 | 0.68 | 0.917 | 0.597 | Up |
| TG(47:0) | 0.820 | 0.069 | 0.002 | 0.685~0.955 | 0.76 | 0.833 | 0.593 | Down |
| PE(36:2) | 0.803 | 0.081 | 0.003 | 0.645~0.961 | 0.88 | 0.750 | 0.630 | Down |
| PE(34:4) | 0.800 | 0.073 | 0.004 | 0.658~0.942 | 0.64 | 0.917 | 0.557 | Down |
| Top7 combined | 1.000 | 0 | 0 | 1.000 | 1.000 | 1.000 | 1.000 | — |
| diagnosis | | | | | | | | |

Further, the High group and the Mid group were used as disease groups, which were recorded as HM groups; and the diagnostic values of the obtained lipids were evaluated by performing ROC analysis on an HM_vs_Norm comparison group. The selection standard was AUC>0.89, P<0.05, among 20 lipids obtained, 6 lipids had higher diagnostic power, and thus might be used as biomarkers, referring to Table 9.

TABLE 9
ROC analysis and combined diagnosis of 6 lipid biomarkers
obtained from HM_vs_Norm comparison group.

| | | SEM | | | | | Best cutoff | |
|---|---|---|---|---|---|---|---|---|
| Lipid | AUC | (±) | P-value | 95% CI | Sensitivity | Specificity | value | Trend |
| TG(52:5) | 0.894 | 0.063 | 0 | 0.777~1 | 0.889 | 0.111 | 0.722 | Up |
| PE(34:4) | 0.926 | 0.047 | 0 | 0.835~1 | 0.833 | 0.917 | 0.750 | Down |
| TG(47:0) | 0.921 | 0.047 | 0 | 0.828~1 | 0.889 | 0.833 | 0.722 | Down |
| PC(42:6) | 0.907 | 0.059 | 0 | 0.793~1 | 0.833 | 1.000 | 0.833 | Down |
| LPC(20:2) | 0.898 | 0.056 | 0 | 0.788~1 | 0.889 | 0.833 | 0.722 | Down |
| PC(O-32:3) | 0.894 | 0.063 | 0 | 0.769~1 | 0.833 | 0.917 | 0.750 | Down |
| Top6 combined | 1.000 | 0 | <0.001 | 1.000 | 1.000 | 1.000 | 1.000 | — |
| diagnosis | | | | | | | | |

Further, according to laboratory research experience, low-grade fatty liver might be restored to a normal state by means of feeding adjustment, such that the High group and the Mid group were used as the disease groups, which were recorded as the HM groups; the Low group and the Norm group were used as basically-normal groups, which were recorded as LN groups; and the diagnostic values of the obtained lipids were evaluated by performing ROC analysis on an HM_vs_LN comparison group. The selection standard was AUC>0.85, P<0.05, among 20 lipids obtained, 5 lipids had higher diagnostic power, and thus might be used as biomarkers, referring to Table 10.

TABLE 10
ROC analysis and combined diagnosis of 5 lipid biomarkers
obtained from HM_vs_LN comparison group.

| | | SEM | | | | | Best cutoff | |
|---|---|---|---|---|---|---|---|---|
| Lipid | AUC | (±) | P-value | 95% CI | Sensitivity | Specificity | value | Trend |
| TG(47:0) | 0.915 | 0.046 | 0 | 0.825~1.000 | 0.778 | 0.947 | 0.725 | Down |
| PE(34:4) | 0.906 | 0.048 | 0 | 0.813~1.000 | 0.833 | 0.895 | 0.728 | Down |
| PC(O-32:3) | 0.883 | 0.063 | 0 | 0.760~1.000 | 0.833 | 0.895 | 0.728 | Down |
| LPC(28:0) | 0.871 | 0.059 | 0 | 0.755~0.988 | 0.833 | 0.842 | 0.675 | Down |
| PC(26:0) | 0.860 | 0.062 | 0 | 0.739~0.981 | 0.833 | 0.789 | 0.623 | Down |
| Top5 combined | 0.971 | 0.026 | 0 | 0.920~1.000 | 1.000 | 0.895 | 0.895 | — |
| diagnosis | | | | | | | | |

Further, in order to distinguish the High group and other states, the Mid group, the Low group, and the Norm group were combined as one group, which was recorded as an MLN group; and the diagnostic values of the obtained lipid biomarkers were evaluated by performing ROC curve screening analysis on an High_vs_MLN comparison group. The selection standard was AUC>0.85, P<0.05, among 20 lipids obtained, 7 lipids had higher diagnostic power, and thus might be used as biomarkers, referring to Table 11.

TABLE 11
ROC analysis and combined diagnosis of 7 lipid biomarkers
obtained from High_vs_MLN comparison group.

| | | SEM | | | | | Best cutoff | |
|---|---|---|---|---|---|---|---|---|
| Lipid | AUC | (±) | P-value | 95% CI | Sensitivity | Specificity | value | Trend |
| LPC(20:2) | 0.909 | 0.062 | 0 | 0.787~1.000 | 0.778 | 1.000 | 0.778 | Down |
| LPC(20:0) | 0.893 | 0.054 | 0 | 0.787~0.998 | 0.889 | 0.821 | 0.710 | Down |
| LPC(17:0) | 0.881 | 0.055 | 0.001 | 0.773~0.989 | 1.000 | 0.714 | 0.714 | Down |
| TG(47:0) | 0.881 | 0.057 | 0.001 | 0.769~0.993 | 1.000 | 0.679 | 0.679 | Down |
| PC(O-32:3) | 0.861 | 0.060 | 0.001 | 0.744~0.979 | 1.000 | 0.750 | 0.750 | Down |
| Cer(d18:0/22:0) | 0.857 | 0.092 | 0.001 | 0.676~1.000 | 0.667 | 1.000 | 0.667 | Down |
| LPE(20:5) | 0.857 | 0.069 | 0.001 | 0.722~0.992 | 0.889 | 0.679 | 0.567 | Down |
| Top7 combined | 1.000 | <0.001 | <0.001 | 1.000 | 1.000 | 1.000 | 1.000 | — |
| diagnosis | | | | | | | | |

A method for diagnosing the conditions of the periparturient dairy cows using the lipid biomarkers of the present disclosure included the following operation.

The fat content in the serum of the periparturient dairy cows was tested by means of a mass spectrometry method; the diagnostic value of a certain obtained lipid biomarker might be evaluated through the ROC analysis result; and determination might be performed in conjunction with whether the trend of the lipid at different stages before and after was the same as the trend listed in the table.

### Embodiment 7: Distribution of Corresponding Lipid Biomarkers in Each Combined Comparison Group

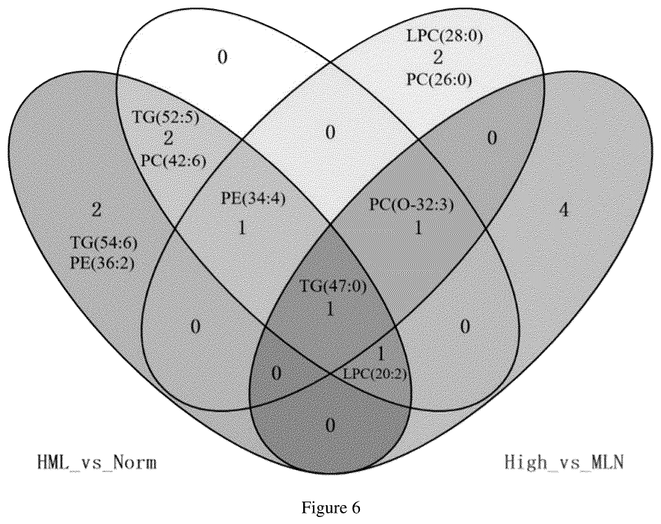

The distribution of the obtained 20 lipid biomarkers having higher diagnostic power in 4 combined comparison groups was shown in FIG. 6. The 4 combined comparison groups were an HML_vs_Norm combined comparison group, an HM_vs_Norm combined comparison group, an HM_vs_LN combined comparison group, and a High_vs_MLN combined comparison group.

### Embodiment 8: Value Description of 20 Lipids

The present disclosure had the following effects. 20 lipid molecules of Cer(d18:0/22:0), DAG(34:1), ePE(36:4), LPC(17:0), LPC(20:0), LPC(20:2), LPC(28:0), LPE(20:5), PC(26:0), PC(42:6), PC(O-30:0), PC(O-32:3), PE(34:4), PE(36:2), PE(36:3), TG(47:0), TG(52:5), TG(52:6), TG(54:6), and TG(56:5) in dairy cow serum samples might be used singly or in combination for distinguishing the health status of the periparturient dairy cows with fatty liver and the health status of healthy dairy cows; and in the Low_vs_Norm comparison group, the Mid_vs_Norm comparison group, the High_vs_Norm comparison group, the High_vs_Mid comparison group, the Mid_vs_Low comparison group, the HML_vs_Norm combined comparison group, the HM_vs_Norm combined comparison group, the HM_vs_LN combined comparison group, and the High_vs_MLN combined comparison group, the corresponding lipids had excellent distinguishing effects, and had the characteristics of high specificity and high sensitivity. Therefore, the lipids had very important practical significance on diagnosing the periparturient dairy cows with fatty liver, and thus had good application prospects.

### Embodiment 9: Other Population Validation—Diagnosis of the Conditions Periparturient Dairy Cows with High-Grade Fatty Liver Using the Identified Lipid Biomarkers of the Present Disclosure

10 (High group) periparturient dairy cows diagnosed with high-grade fatty liver through liver biopsy and 10 (Norm group) healthy periparturient dairy cows were selected, and the serum of each dairy cow at fasting was acquired.

The lipids PC(42:6), LPC(17:0), PC(O-32:3), LPC(20:2), LPC(20:0), TG(47:0), PE(34:4), and TG(52:5) for diagnosing high-grade fatty liver in the periparturient dairy cows were screened according to Embodiment 6 of the present disclosure.

The fat content in the serum of the 20 dairy cows was tested with an UPLC-TQMS, and results were shown as follows.

Comparison between the Norm group and the High group:

The content of the lipids PC(42:6), LPC(17:0), PC(O-32:3), LPC(20:2), LPC(20:0), TG(47:0), and PE(34:4) was reduced, and the content of the lipid TG(52:5) was increased, which were consistent with diagnostic analysis in Embodiment 6 of the present disclosure, which indicated that the lipid biomarkers screened in the present disclosure could be used for testing the fatty liver in the periparturient dairy cows, and had high clinical diagnostic application potential values.

The above are only the preferred embodiments of this application and are not intended to limit this application. For those skilled in the art, this application may have various modifications and variations. Any modifications, equivalent replacements, improvements, and the like made within the spirit and principle of this application shall fall within the scope of protection of this application.

What is claimed is:

1. A lipid in diagnosis and early warning of fatty liver in periparturient dairy cows as a biomarker, wherein the lipid is one or more lipids of the following (1)-(8):
(1) Ceramide (Cer) (d18:0/22:0);
(2) Diacylglycerol (DAG) (34:1);
(3) ether Phosphatidyl Ethanolamine (ePE) (36:4);
(4) Lysophosphatidylcholine (LPC);
(5) Lysophosphatidylethanolamine (LPE) (20:5);
(6) Phosphatidylcholine (PC);
(7) Phosphatidylethanolamine (PE); or
(8) Triglyceride (TG).

2. The lipid according to claim 1, wherein the LPC is selected from LPC(17:0), LPC(20:0), LPC(20:2), or LPC(28:0);
the PC is selected from PC(26:0), PC(42:6), PC(O-30:0), or PC(O-32:3);
the PE is selected from PE(34:4), PE(36:2), or PE(36:3); and
the TG is selected from TG(47:0), TG(52:5), TG(52:6), TG(54:6), or TG(56:5).

3. The lipid according to claim 1, wherein the lipid is selected from one or more of TG(52:5), TG(56:5), TG(54:6), or TG(52:6), when the lipid is a biomarker for diagnosing low-grade fatty liver in periparturient dairy cows.

4. The lipid according to claim 1, wherein the lipid is selected from one or more of PE(36:3), PE(36:2), PE(34:4), TG(52:5), TG(47:0), PC(26:0), or TG(56:5), when the lipid is a biomarker for diagnosing mid-grade fatty liver in periparturient dairy cows as a biomarker.

5. The lipid according to claim 1, wherein the a lipid is selected from one or more of PC(42:6), LPC(17:0), PC(O-32:3), LPC(20:2), LPC(20:0), TG(47:0), PE(34:4), or TG(52:5), when the lipid is a biomarker for diagnosing high-grade fatty liver in periparturient dairy cows as a biomarker.

6. The lipid according to claim 1, wherein the lipid is selected from one or more of LPC(20:2), Cer(d18:0/22:0), LPC(20:0), or LPE(20:5), when the lipid is a biomarker for distinguishing dairy cows with high-grade fatty liver and dairy cows with mid-grade fatty liver in a periparturient period as a biomarker.

7. The lipid according to claim 1, wherein the lipid is selected from one or more of ePE(36:4), LPC(28:0), PC(O-30:0), DAG(34:1), or PC(26:0), when the lipid is a biomarker for distinguishing dairy cows with mid-grade fatty liver and dairy cows with low-grade fatty liver in a periparturient period.

8. The lipid according to claim 1, wherein the lipid is a lipid in the following (1)-(2) used singly or in combination:
(1) TG(52:5), PC(42:6), LPC(20:2), TG(54:6), TG(47:0), PE(36:2), or PE(34:4); or
(2) TG(52:5), PE(34:4), TG(47:0), PC(42:6), LPC(20:2), or PC(O-32:3), when the lipid is a biomarker for distinguishing dairy cows with fatty liver and healthy dairy cows in a periparturient period as a biomarker.

9. A reagent for testing at least one lipid according to claim 1 in preparation of products for non-invasive recognition of fatty liver disease in periparturient dairy cows.

10. The reagent according to claim 9, wherein the reagent is a reagent for testing a lipid in the serum of dairy cows, or a reagent for directly testing a lipid in a blood sample.

11. A method for diagnosing conditions of periparturient dairy cows, wherein the method comprises: detecting the fat content in the serum of the periparturient dairy cows by means of a mass spectrometry method;
evaluating the diagnostic value of a lipid biomarker through the ROC analysis; and
determining the conditions of the periparturient dairy cows in conjunction with whether the change trend of the lipid at different stages before and after is the same as the change trend in the positive controls;
wherein the lipid biomarker is selected from one or more of the following:
(1) Ceramide (Cer) (d18:0/22:0);
(2) Diacylglycerol (DAG) (34:1);
(3) ether Phosphatidyl Ethanolamine (ePE) (36:4);
(4) Lysophosphatidylcholine (LPC);
(5) Lysophosphatidylethanolamine (LPE) (20:5);
(6) Phosphatidylcholine (PC);
(7) Phosphatidylethanolamine (PE); or
(8) Triglyceride (TG).

12. The method according to claim 11, wherein the LPC is selected from LPC(17:0), LPC(20:0), LPC(20:2), or LPC(28:0);
the PC is selected from PC(26:0), PC(42:6), PC(O-30:0), or PC(O-32:3);
the PE is selected from PE(34:4), PE(36:2), or PE(36:3); and
the TG is selected from TG(47:0), TG(52:5), TG(52:6), TG(54:6), or TG(56:5).

13. The method according to claim 11, wherein when the lipid is selected from one or more of (1) or (2):
(1) TG(52:5), PC(42:6), LPC(20:2), TG(54:6), TG(47:0), PE(36:2), or PE(34:4); or
(2) TG(52:5), PE(34:4), TG(47:0), PC(42:6), LPC(20:2), or PC(O-32:3).

14. The method according to claim 11, wherein when the lipid is selected from one or more of ePE(36:4), LPC(28:0), PC(O-30:0), DAG(34:1) or PC(26:0), the method is a method for distinguishing dairy cows with mid-grade fatty liver and dairy cows with low-grade fatty liver in a periparturient period.

15. The method according to claim 11, wherein when the lipid is selected from one or more of LPC(20:2), Cer(d18:0/22:0), LPC(20:0), or LPE(20:5), the method is a method for distinguishing with high-grade fatty liver and dairy cows with mid-grade fatty liver in a periparturient period.

16. The method according to claim 11, wherein when the lipid is selected from one or more of PC(42:6), LPC(17:0), PC(O-32:3), LPC(20:2), LPC(20:0), TG(47:0), PE(34:4), or TG(52:5), the method is a method for diagnosing high-grade fatty liver in periparturient dairy cows.

17. The method according to claim 11, wherein when the lipid is selected from one or more of PE(36:3), PE(36:2), PE(34:4), TG(52:5), TG(47:0), PC(26:0), or TG(56:5), the method is a method for diagnosing mid-grade fatty liver in periparturient dairy cows.

18. The method according to claim 11, wherein when the lipid is selected from one or more of TG(52:5), TG(56:5), TG(54:6), or TG(52:6), the method is a method for diagnosing low-grade fatty liver in periparturient dairy cows.
